(19) United States
(12) Patent Application Publication
(10) Pub. No.: US 2024/0038349 A1
(43) Pub. Date: Feb. 1, 2024

(54) Systems and methods for explaining output from a machine learning model

(71) Applicant: pulseData, Inc., New York, NY (US)

(72) Inventors: Larson Shih, Stamford, CT (US); Chris Kipers, Pomfret Center, NY (US); Teddy Cha, Brooklyn, NY (US); Job Evers, Somerville, MA (US); Tia Yue Yu, Irvine, CA (US); Annalee Kuse, New York, NY (US); John Li, Santa Monica, CA (US); Hai Po Sun, New York, NY (US)

(21) Appl. No.: 18/226,808

(22) Filed: Jul. 27, 2023

Related U.S. Application Data
(60) Provisional application No. 63/369,942, filed on Jul. 30, 2022.

(51) Int. Cl. G16H 15/00; G16H 50/30; G16H 50/70; G16H 10/60

(52) U.S. Cl. CPC G16H 15/00; G16H 50/30; G16H 50/70; G16H 10/60

(57) ABSTRACT

Methods and systems for explaining an output from a machine learning model. The methods include receiving from a machine learning model a probability score, a plurality of features, and a contribution value for each feature; transforming the plurality of features to a reduced number of features; generating at least one cluster of a portion of a population; grouping the patient in the generated cluster; and visually presenting to a user a comparison of the reduced number of features of the profile with the plurality of features associated with the portion of the population to explain the probability score.

### CROSS-REFERENCE TO RELATED APPLICATIONS

The present application claims the benefit of and priority to U.S. provisional application No. 63/369,942, filed on Jul. 30, 2022, the entire disclosure of which is incorporated by reference as if set forth in its entirety herein.

### TECHNICAL FIELD

Embodiments described herein generally relate to systems and methods for processing machine learning model outputs and, more particularly but not exclusively, to systems and methods for explaining the reason(s) behind machine learning model outputs.

### BACKGROUND

The process of providing healthcare to patients produces large amounts of data. As the number of health devices for monitoring a patient increases in the coming years, the amount of data will further increase.

Existing electronic health record (EHR) interfaces are most suited for inputting data, such as data associated with lab orders, notes, and medication suggestions. However, these existing EHR interfaces may not be as suitable for providing a useful or informative review of a patient or their condition(s). This makes reviewing patient data difficult, and may increase the likelihood of overlooking details associated with the patient's health.

This can be particularly true in reviewing the results from machine learning models. While machine learning models may uncover patterns unnoticed by a human reviewer, the model's mapping of inputs to outputs is often not explainable. Explaining the values outputted by a machine learning model can therefore be difficult, and a user may not fully understand the reasoning behind an output. This is particularly true in healthcare settings, in which it is desirable to have a thorough understanding of a patient's health. However, explaining values outputted by machine learning models can be difficult in any other type of application.

A need exists, therefore, for improved systems and methods for explaining machine learning model outputs.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a method for explaining an output from a machine learning model. The method includes receiving from a machine learning model a probability score associated with a profile, a plurality of features associated with the profile, and a contribution value for each of the plurality of features that represents each feature's contribution to the probability score; transforming the plurality of features to a reduced number of features based at least on the contribution value of each of the plurality of features; generating at least one cluster of a portion of a population, wherein the cluster is generated based on a probability score associated with the portion of the population, a plurality of features associated with the portion of the population, and a contribution value for each of the plurality of features associated with the portion of the population; grouping the profile in the generated cluster based on the profile contribution values and the contribution values of the portion of the population; and visually presenting to a user a comparison of the reduced number of features of the profile with the plurality of features associated with the portion of the population to explain the probability score.

In some embodiments, the method further includes graphically presenting the probability score to the user. In some embodiments, the visual presentation includes a graphical comparison of the reduced number of features of the profile with the plurality of features associated with the portion of the population. In some embodiments, the method further includes generating a plurality of clusters of portions of the population, wherein the graphical display includes a comparison of the reduced number of features of the profile with the plurality of features associated with the plurality of clusters of portions of the population.

In some embodiments, the profile is associated with a patient, the plurality of features relate to healthcare data associated with the patient, and the probability score is a phenotype prediction.

In some embodiments, visually presenting the comparison includes presenting at least one of at least one of a radar chart, a sliding scale graph, or a distribution graph.

In some embodiments, the profile is grouped in the cluster based on the profile contribution values and the contribution values of the portion of the population being similar to each other within a threshold.

In some embodiments, determining the probability score includes making a statistical comparison between at least one feature associated with the profile and a corresponding feature for the portion of the population.

According to another aspect, embodiments relate to a system for explaining an output from a machine learning model. The system includes an interface for at least receiving from a machine learning model a probability score associated with a profile, a plurality of features associated with the profile, and a contribution value for each of the plurality of features that represents each feature's contribution to the probability score; at least one processor executing instructions stored on memory and configured to transform the plurality of features to a reduced number of features of based at least on the contribution value of each of the plurality of features, generate at least one cluster of a portion of a population, wherein the cluster is generated based on a probability score associated with the portion of the population, a plurality of features associated with the portion of the population, and a contribution value for each of the plurality of features associated with the portion of the population; group the profile in the generated cluster based on the profile contribution values and the contribution values of the portion of the population; and visually present to a user a comparison of the reduced number of features of the profile with the plurality of features associated with the portion of the population to explain the probability score.

In some embodiments, the system further includes a user interface for graphically presenting the probability score to the user. In some embodiments, the visual presentation includes a graphical comparison of the reduced number of features of the profile with the plurality of features associated with the portion of the population. In some embodiments, the processor is further configured to generate a plurality of clusters of portions of the population, wherein the graphical display includes a comparison of the reduced number of features of the profile with the plurality of features associated with the plurality of clusters of portions of the population.

In some embodiments, the profile is associated with a patient, the plurality of features relate to healthcare data associated with the patient, and the probability score is a phenotype prediction.

In some embodiments, the comparison is visually presented as at least one of a radar chart, sliding scale graph, or a distribution graph.

In some embodiments, the at least one processor groups the profile in the cluster based on the profile contribution values and the contribution values of the portion of the population being similar to each other within a threshold.

In some embodiments, the processor determines the probability score by making a statistical comparison between at least one feature associated with the profile and a corresponding feature for the portion of the population.

According to yet another aspect, embodiments relate to a computer program product for explaining an from a machine learning model. The computer program product comprises computer executable code embodied in one or more non-transitory computer readable media that, when executing on one or more processors, performs the steps of receiving from a machine learning model a probability score associated with a profile, a plurality of features associated with the profile, and a contribution value for each of the plurality of features that represents each feature's contribution to the probability score; transforming the plurality of features to a reduced number of features of based at least on the contribution value of each of the plurality of features; generating at least one cluster of a portion of a population, wherein the cluster is generated based on a probability score associated with the portion of the population, a plurality of features associated with the portion of the population, and a contribution value for each of the plurality of features associated with the portion of the population; grouping the profile in the generated cluster based on the profile contribution values and the contribution values of the portion of the population; and visually presenting to a user a comparison of the reduced number of features of the profile with the plurality of features associated with the portion of the population to explain the probability score.

In some embodiments, the computer program product further includes computer executable code that, when executing on one or more processors, performs the step of graphically presenting the probability score to the user. In some embodiments, the visual presentation includes a graphical comparison of the reduced number of features of the profile with the plurality of features associated with the portion of the population. In some embodiments, the computer program product further includes computer executable code that, when executing on one or more processors, performs the step of generating a plurality of clusters of portions of the population, wherein the graphical display includes a comparison of the reduced number of features of the profile with the plurality of features associated with the plurality of clusters of portions of the population.

### BRIEF DESCRIPTION OF DRAWINGS

Non-limiting and non-exhaustive embodiments of this disclosure are described with reference to the following figures, wherein like reference numerals refer to like parts throughout the various views unless otherwise specified.

FIG. 1 illustrates a system for explaining an output from a machine learning model in accordance with one embodiment;

FIG. 2 presents a table of features, values, and associated contribution values in accordance with one embodiment;

FIG. 3 presents a table showing how grouped features of a patient compare to those of a cluster in accordance with one embodiment;

FIG. 4 illustrates a user interface presenting a comparison chart in accordance with one embodiment;

FIG. 5 illustrates a user interface presenting a plurality of sliding scale graphs in accordance with one embodiment;

FIGS. 6A-F present distribution graphs for various features in accordance with one embodiment; and

FIG. 7 depicts a flowchart of a method for explaining an output from a machine learning model in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The term “tangible computer-readable storage medium” shall accordingly be taken to include, but not be limited to: solid-state memories such as a memory card or other package that houses one or more read-only (non-volatile) memories, random access memories, or other re-writable (volatile) memories, a magneto-optical or optical medium such as a disk or tape, or other tangible media which can be used to store information. Accordingly, the disclosure is considered to include any one or more of a tangible computer-readable storage medium, as listed herein and including art-recognized equivalents and successor media, in which the software implementations herein are stored.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

One of the ways to explain a patient cohort is to present the patient in relation to a “prototype cluster.” This is often done using test results, such that a patient is grouped in a cluster with other patients that have similar test results. However, using test results only does not provide a thorough, comprehensive, or otherwise meaningful understanding of a patient's health.

Machine learning models may highlight patterns that are unnoticed by a user using only test results. However, the mapping of inputs to outputs in machine learning pipelines is often not explainable. A user such as a reviewing clinician may not fully understand the reasoning behind a model output. For example, a user may not fully understand which features or medical parameters associated with a patient contributed to a phenotype prediction or the extent of any contribution.

Existing models for analyzing healthcare data may output normalized values that ultimately do not provide a user with a thorough understanding of a patient's health. For example, human weight is normally expressed in pounds (lbs). A model may normalize a patient's weight to a score between −1 to 1, where the value of zero (0) indicates the patient's weight is average. Accordingly, a user may receive normalized values as opposed to medically meaningful, raw values.

Oftentimes, a model may produce an output that is dependent on several of these features. These may include negative values and positive values. The net effect of these values depicts the model's output. However, these outcomes do not provide a user with any additional insight regarding a patient's health, such as the reasoning behind the output.

The embodiments described herein provide novel techniques for explaining an output from a machine learning model. Although the present application largely discusses the embodiments in conjunction with healthcare and for patient treatment, the features of the embodiments herein may be implemented in any other applications reliant on machine learning models to generate one or more outputs based on inputted features. For example, companies or marketing agencies may analyze features associated with prospective customers or target demographics to better understand why a particular customer is predicted to respond favorably or unfavorably to a product, service, or advertisement campaign.

As another example, the embodiments herein may be used in conjunction with models for analyzing financial or commodity markets. Oftentimes models analyze several features associated with a company's stock price to predict what the price of the stock will be in the future. These models may be based on several company- or stock pattern-specific features such as revenue, profit, cash flow, moving average(s), relative strength index, moving average convergence/divergence (MACD), volume, etc. Accordingly, the embodiments herein described herein may analyze the output of the model(s), perform any required dimension reduction and clustering steps described herein to, for example, provide easy-to-understand explanations of a model output.

Other applications may include those in weather analysis, military strategy formation and planning, traffic analysis, farming, or the like. Accordingly, the features of the described embodiments may be implemented in any other application in addition to or in lieu of those described herein.

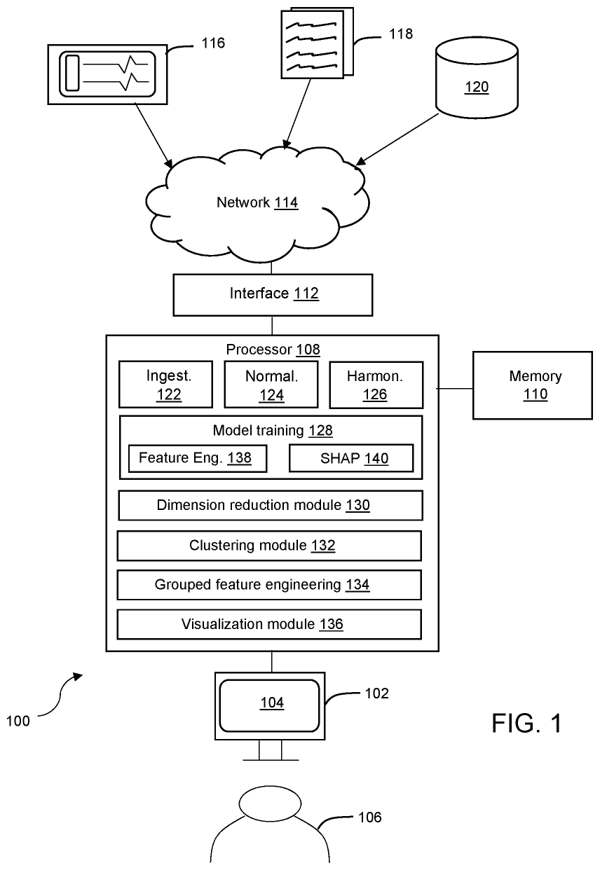

FIG. 1 illustrates a system 100 for explaining an output from a machine learning model in accordance with one embodiment. The system 100 may include a user device 102 executing a user interface 104 accessible by a user 106. The user 106 may include healthcare personnel such as a treating clinician, for example.

The user device 102 may be any sort of device that can visually present data. For example, in some embodiments, the user device may be a personal computer (PC), laptop, tablet, smartphone, television, smartwatch, virtual- or augmented-reality display, etc. This list of devices is exemplary, and other devices whether available now or invented hereafter may be used in conjunction with the embodiments herein.

The user device 102 may be in operable connectivity with one or more processors 108 executing instructions stored on memory 110. The processor(s) 108 may be any hardware device capable of executing instructions stored on memory 110 to provide various components, modules, or services. The processor 108 may include a microprocessor, a field programmable gate array (FPGA), an application-specific integrated circuit (ASIC), or other similar devices.

In some embodiments, such as those relying on one or more ASICs, the functionality described as being provided in part via software may instead be configured into the design of the ASICs and, as such, the associated software may be omitted. The processor(s) 108 may be configured as part of the user device 102 (e.g., a PC) or located at some remote location.

The memory 110 may be L1, L2, L3 cache, or RAM memory configurations. The memory 110 may include non-volatile memory such as flash memory, EPROM, EEPROM, ROM, and PROM, or volatile memory such as static or dynamic RAM, as discussed above. The exact configuration/type of memory 110 may of course vary as long as instructions for processing an output from a machine learning model be performed by the system 100.

The system 100 may also include an interface 112 to receive healthcare data over one or more networks 114. The network(s) 114 may link the various components with various types of network connections. The network(s) 114 may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks 114 may also comprise, include, or interface to any one or more of a Wireless Application Protocol (WAP) link, a Wi-Fi link, a microwave link, a General Packet Radio Service (GPRS) link, a Global System for Mobile Communication (GSM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The interface 112 may receive healthcare data from a variety of data sources over the network(s) 114. This healthcare data may relate to or otherwise be obtained through treatment of one or more patients. The sources of the healthcare data may include, but are not limited, patient monitoring devices 116, clinical notes 118 (whether made electronically or on paper), databases 120 such as those storing EHRs, or the like.

The one or more processor(s) 108 may include or otherwise execute one or more services, components, or modules for analyzing healthcare data, modeling the healthcare data, and visually presenting data associated with model output(s). These modules may include, but are not limited to, an ingestion module 122, normalization module 124, harmonization module 126, model training module 128, dimension reduction module 130, clustering module 132, grouped feature engineering module 134, and a visualization module 136.

In operation, the ingestion module 122 may receive from the interface 112 healthcare data related to a patient. For example, the healthcare data may include, but is not limited to, lab test results, billing data, clinical laboratory medication history, pharmacy data, biometrics, vitals, clinical notes, audio or textual inputs, data generated from electronic health records (EHR) systems, or the like. In some embodiments, the ingestion module 122 may receive this data as part of a training phase for generating supervised machine learning models.

As the healthcare data may be received from several different sources, the healthcare data may include data in a variety of formats. For example, and without limitation, the healthcare data may include text files, audio files, CSV files, digital data, or the like.

The normalization module 124 may execute instructions stored on memory 110 to perform one or more procedures to normalize the received healthcare data. This ensures that all data is represented in the same format. For example, data represented in units of “lbs,” “LBs,” and “pounds” may be converted to “lbs.” Similarly, the memory 110 may store conversion factors between units to enable the normalization module 124 to execute any appropriate unit conversion procedures (e.g., to convert kilograms to pounds).

The harmonization module 126 may execute instructions stored on memory 110 to ensure all data is on the same scale. For example, if healthcare data associated with a patient includes an “age” attribute with an associated value of “0” (zero), and a “weight” attribute with a value of “3,000” the meaning of these values may not be readily apparent to a user. However, an age value of “0” may suggest that the patient is a newborn child. In this scenario, the harmonization module 126 may recognize that a weight value of, e.g., 2,500 to 5,000 may be in grams as those are typical weights of newborn children. The harmonization module 126 may then perform any unit conversion procedures to, for example and if necessary, convert the patient's weight to lbs.

The model training module 128 may train and execute one or more different types of machine learning models to generate an output for a patient (e.g., the probability the patient has a particular disease). The model(s) may be trained on hundreds or thousands of possible features or variables (for simplicity, “features”). The embodiments described herein may execute models such as those based on linear regression, random forest decision trees, support vector machines (SVMs), or any other type of model whether available now or formulated hereafter. For example, the embodiments described herein may execute models such as those described in Applicant's co-pending U.S. patent application Ser. No. 18/219,168, filed on Jul. 7, 2023, the content of which is incorporated by reference as if set forth in its entirety herein.

The model training module 128 may also output a risk score associated with a predicted phenotype. For example, the risk score may represent the probability that a patient has a predicted phenotype, or may represent the severity of the predicted phenotype. The risk score (also referred to as a “probability score”) may be based on the features present in the patient healthcare data, the number of models outputting a positive prediction, or some combination thereof.

The model training module 128 may review the statistical models to assess their performance. The model training module 128 may use a holdout data set to evaluate the performance of the generated model(s). In these cases, the holdout dataset may include data that was not used to generate the model(s) but may include similar data features. In some embodiments, the holdout data may include the most recently-gathered healthcare data.

The model training module 128 may further include a feature engineering submodule 138 and a Shapley Additive Explanations values (SHAP) submodule 140. The feature engineering submodule 138 may identify variables or combinations of variables that are likely predictive of a condition. For example, the feature engineering submodule 138 may perform one or more procedures involving identifying, extracting, transforming, or otherwise manipulating raw data to select the feature(s) that can most accurately predict a condition. In other words, the feature engineering submodule 138 may select a certain number of features based on their significance to a model's accuracy. For example, in some embodiments, the feature engineering submodule 138 may identify 40-50 features from hundreds or thousands of features.

The SHAP submodule 140 may then identify the SHAP value of each of the selected features. The SHAP values represents the contribution of each feature toward a given prediction or the relative importance of each feature in contributing to an overall probability score.

SHAP values may be negative or positive. Accordingly, these values may cancel out each other, and the net effect of the values may determine the probability score associated with a patient.

FIG. 2 presents a table 200 of features, values, and associated SHAP values in accordance with one embodiment. These values may be associated with a patient being examined for Chronic Kidney Disease (for simplicity, “CKD”), for example.

As seen in FIG. 2, the SHAP values may range in magnitude and have positive or negative values. The magnitude of each value may indicate the amount by which the feature's value contributes to the patient's score. For example, the SHAP value of 0.28 indicates that the value of 161 (corresponding the number of days since their last acidosis), contributes to the patient's score positively. Similarly, the SHAP value of 0.16 indicates that the value of 54 (corresponding to the patient's acute kidney injury (AKI) count), also contributes to the patient's score positively. However, as the SHAP value 0.28 is greater than 0.16, the “number of days since last acidosis” feature and associated value has a greater effect on the predicted score than the patient's AKI count and associated value. If, on the other hand, the patient's “days since last acidosis” had a value that was less than 161, the associated SHAP value may also be less.

Some of these values may cancel out other values, and the net effect of the features may be a probability score associated with a patient. For example, table 200 includes negative values corresponding to the features CKD stage (−0.19), CKD S4 180D monthly percentage (−0.14), and CKD S4 180 D Monthly History Percentage (−0.13). Although the SHAP values associated with the majority feature values are positive, these negative values may reduce or otherwise have a negative effect on the probability score.

As with the positive values discussed above, the amount by which these feature values affect the patient's score is a function of the associated SHAP value. That is, the value for CKD stage (3) will have a greater impact on the patient's score than the value for the feature “CKD S4 180D Monthly History Percentage.”

Accordingly, the output of the model training module 128 may be, for each of one or more patients, a list of features, a value for each feature, and a SHAP value for each feature. These outputs may then be communicated to the dimension reduction module 130.

The dimension reduction module 130 may then group the received features into a smaller number of features that are medically and/or statistically meaningful. In other words, the dimension reduction module 130 reduces the number of features analyzed in subsequent steps.

The system 100 may compare the data of table 200 for a particular patient with that of other patients. For example, the database(s) 120 may store data feature values and associated SHAP values for a patient population (e.g., of hundreds or thousands of patients).

For example, the dimension reduction module 130 may reduce the features of FIG. 2 into dimensions of (1) Age (not shown in table 2), (2) CKD stage, (3) days since last AKI, (4) AKI count, (5) chronic conditions, and (6) signs and symptoms. A single dimension may correspond to several related or interacting features. For example, the “signs and symptoms” dimension may include or otherwise be based on several features such as acidosis, hyperkalemia, etc. Other dimensions may be based on a single feature. For example, the age and “days since last AKI” are each based on a single feature.

The clustering module 132 may then compare the SHAP value patterns of the six above dimensions for the patient with those of one or more patient populations. The clustering module 132 may identify patients that do not necessarily have the same values for features, but identify other patients that have similar SHAP values for the same features. In other words, the embodiments herein may identify patients with features that contribute to risk scores in a similar way.

For example, the clustering module 132 may not necessarily group all patients who are 50 years old and have a certain diagnosis. Rather, the clustering module 132 may group together patients whose particular features (such as age, days since last AKI) contribute to their score in similar way (i.e., have similar SHAP values). For example, a first patient that is 50 years old and second patient that is 70 years old may each have a SHAP value of −0.14 for their Age feature. These patients may be clustered together, even though the first patient is 20 years younger than the second patient.

The clustering module 132 may generate any number of clusters and may base the clusters on any particular characteristic(s) of a population. For example, the clustering module 132 may generate one or more clusters that represent patients having varying degrees of risk or otherwise some factor(s) that may affect prescribed treatment or medications.

In some embodiments, the clustering module 132 may generate clusters 1-5, where cluster 1 may include patients who have had a recent acute kidney injury (AKI); cluster 2, patients that have consistently been high risk for CKD; cluster 3, patients that exhibit certain high-risk symptoms of CKD; cluster 4, patients that have had emergency dialysis; and cluster 5, patients that are younger than 40 years old and have moderate-to-high CKD symptoms. These clusters may be based on, for example, medical codes that correspond to a patient's diagnosis or previously-performed procedures.

The grouped feature engineering module 134 may evaluate the dimensionally-reduced features to determine which are clinically relevant and common across multiple generated clusters. From this data, the grouped feature engineering module 134 may generate a representative patient profile that can be compared to one or more clusters.

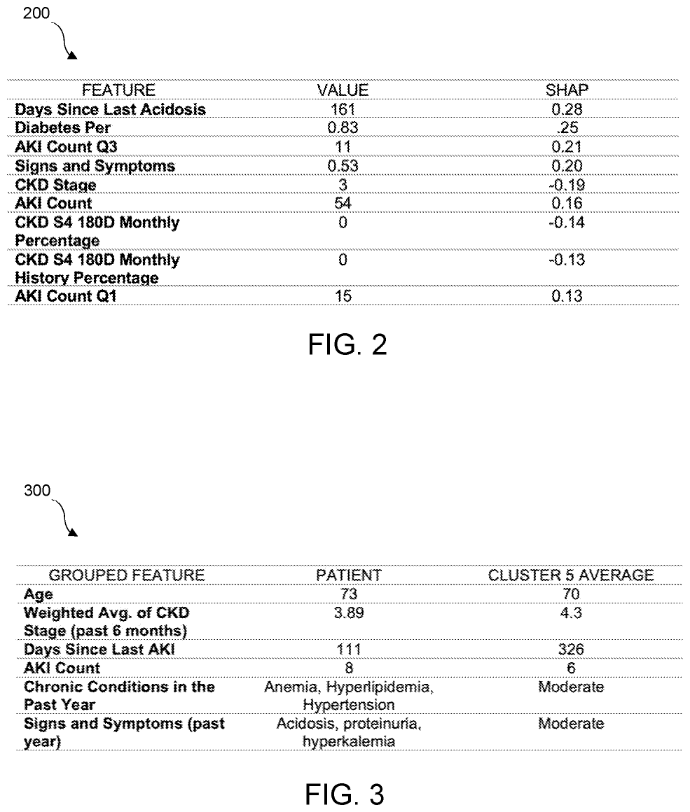

FIG. 3 presents a table 300 showing how the grouped features of the patient compare with those of the average values of cluster 5 (described above) in accordance with one embodiment. The patient may be compared to clusters 1-4, as well, and a table such as the table 300 may be produced for each cluster. In this particular example, the patient may be most closely related with cluster 5.

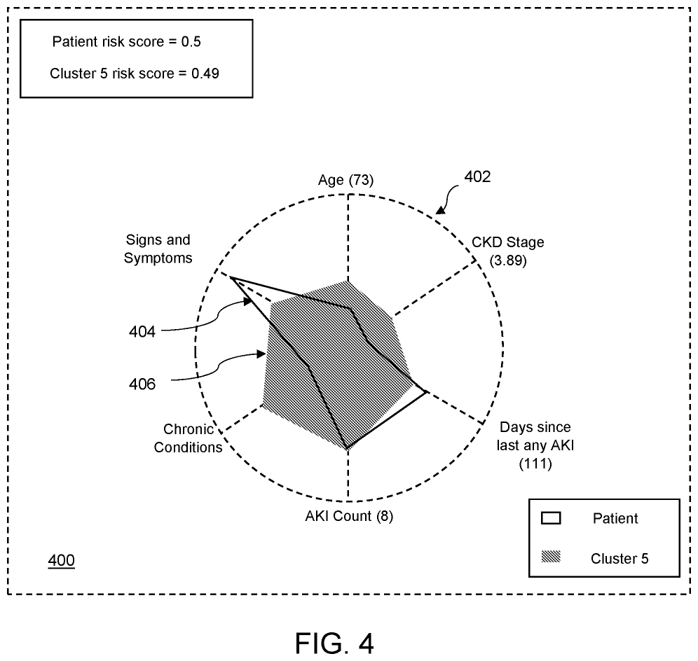

The visualization module 136 may present a comparison of this data via the user interface 104 in a variety of ways. For example, FIG. 4 illustrates a user interface 400 presenting a radar chart 402 that compares the patient profile 404 with the cluster 5 profile 406. The cluster 5 profile 406 is based on the cluster 5 average values of FIG. 3. The “radius” of the patient profile 404 and the radius of the cluster average 406 represents the normalized grouped feature indices based on percentage rank. However, and as mentioned above, the embodiments described herein may similarly compare the patient to each of the other clusters 1-4.

In this case, a user 106 may review the radar chart 402 to gather additional insights about the patient's probability score and causes. For example, the chart 402 shows that the patient's values for “Signs and Symptoms” and “Days Since Last AKI” are driving the patient's risk score (0.05) slightly higher than the risk score associated with cluster 5 (0.49). However, the patient's values for “Age,” “CKD stage,” and “Chronic Conditions” have the opposite effect on the patient's risk score.

The cluster(s) to which a patient is most similar may affect the prescribed treatment(s). For example, the average age of a cluster may be 44 years old and may have an average number of 7 AKI events within the past year. If an analyzed patient is 36 years old and has had 10 AKI events within the past year and is most similar to this cluster, it may affect the treatment plan ultimately assigned to the patient.

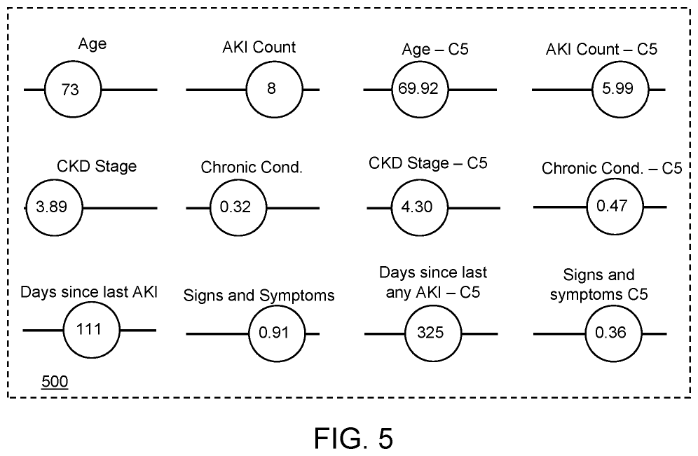

The visualization module 136 herein may present the comparisons between a patient and clusters in a variety of other ways. For example, FIG. 5 illustrates a user interface 500 presenting several sliding scale graphs depicting how a patient's feature indices or values compare to the average of a cluster. As in FIG. 4, FIG. 5 illustrates how the patient's feature indices compare to those of the average of cluster 5 (C5).

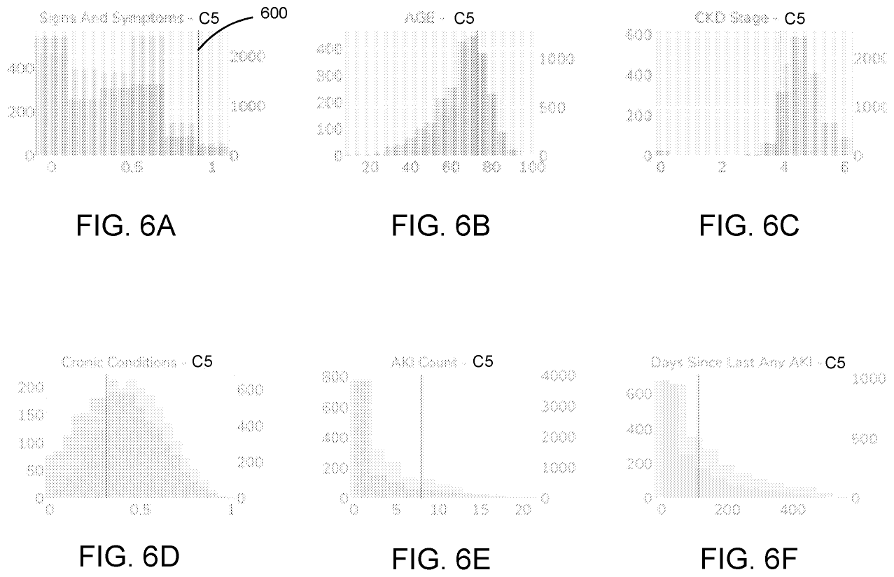

As another example, FIGS. 6A-F presents several distribution graphs illustrating how a particular patient feature compares to those of cluster 5 (C5). Specifically, FIGS. 6A-F show how the patient, represented by vertical line 600, compares to the distribution of each grouped feature set in cluster 5 (illustrated as the light gray bars), and to the distribution of each grouped feature set of the total population (illustrated as dark gray bars). FIGS. 6A-F can be used in several ways. One is to compare the specified cluster (C5) to the total population across the identified grouped feature sets. For example, comparing the average age of the specified cluster (C5) to the average age of the total population. Another is to compare a specific patient to both the specified cluster (C5) and the total population, which can provide insight into the patient's medical profile and risk factors.

The combination of machine learning clustering and the dimension reduction steps of the grouped features provides a systematic framework for analyzing a profile. Additionally, comparing a patient with clusters providers further insights into a patient's probability score that are unavailable through existing model-based techniques.

Existing techniques for analyzing a patient's healthcare data use EHR data, claims data, lab data, or the like to diagnosis a patient. However, these existing techniques do not provide further insight into a patient's health or actionable knowledge for medical personnel. For example, a risk score itself does not tell medical personnel such as a treating nephrologist or care manager why a particular score was calculated. Accordingly, the care manager may desire additional information to assist developing a successful treatment plan.

Similarly, some users or patients may prefer human-readable or human-interpretable reports. The above-described displays or visualization techniques provide results and reasons behind the results in an easy-to-understand manner.

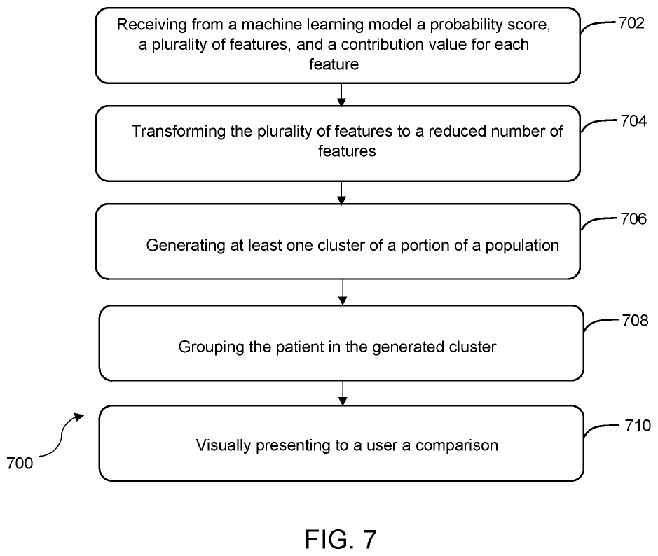

FIG. 7 depicts a flowchart of a method 700 for explaining an output from a machine learning model in accordance with one embodiment. The system 100 of FIG. 1 or components thereof may perform the steps of method 700.

Step 702 involves receiving from a machine learning model a probability score associated with a profile, a plurality of features associated with the profile, and a contribution value for each of the plurality of features that represents each feature's contribution to the probability score. A machine learning model may be tasked with receiving a set of features associated with a profile. For example, in some embodiments, the profile may be a patient profile, and the features may be healthcare data associated with the patient. In this situation, the machine learning model may be tasked with generating a prediction about a patient's health, such as the probability the patient has a particular ailment, disease, phenotype, condition, etc. The features may also be associated with a contribution score that represents their contribution to the score.

Step 704 involves transforming the plurality of features to a reduced number of features based at least on the contribution value of each of the plurality of features. There may a prohibitively high number of features such that a user (e.g., a treating clinician) would have a difficult time in reviewing all features. Accordingly, a dimension reduction service or module such as the dimension reduction module 130 of FIG. 1 may reduce the number of features to a reduced number of features or dimensions. The reduction may be based on the contribution values of features which, in some embodiments, may be or be based on SHAP values. As discussed previously, a single dimension can correspond to several features, or may correspond to only a single feature.

Step 706 involves generating at least one cluster of a portion of a population, wherein the cluster is generated based on a probability score associated with the portion of the population, a plurality of features associated with the portion of the population, and a contribution value for each of the plurality of features associated with the portion of the population. The clusters may be generated based on patterns across SHAP values of patients of a patient population.

Step 708 involves grouping the profile in the generated cluster based on the profile contribution values and the contribution values of the portion of the population. In operation, there may be several clusters that are each associated with patients with specific characteristics. For example, and as discussed previously, there may be a cluster specifically for patients of a certain age and associated with certain medical codes.

Step 710 involves visually presenting to a user a comparison of the reduced number of features of the profile with the plurality of features associated with the portion of the population to explain the probability score. For example, the comparison may be presented as one or more graphical displays. These allow a user to see which features deviate from cluster averages and therefore allows a user to gain a better understanding of a probability score.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for explaining an output from a machine learning model, the method comprising:
receiving from a machine learning model:
a probability score associated with a profile,
a plurality of features associated with the profile, and
a contribution value for each of the plurality of features that represents each feature's contribution to the probability score;
transforming the plurality of features to a reduced number of features based at least on the contribution value of each of the plurality of features;
generating at least one cluster of a portion of a population, wherein the cluster is generated based on:
a probability score associated with the portion of the population,
a plurality of features associated with the portion of the population, and
a contribution value for each of the plurality of features associated with the portion of the population;
grouping the profile in the generated cluster based on the profile contribution values and the contribution values of the portion of the population; and
visually presenting to a user a comparison of the reduced number of features of the profile with the plurality of features associated with the portion of the population to explain the probability score.

2. The method of claim 1 further comprising graphically presenting the probability score to the user.

3. The method of claim 2 wherein the visual presentation includes a graphical comparison of the reduced number of features of the profile with the plurality of features associated with the portion of the population.

4. The method of claim 3 further comprising generating a plurality of clusters of portions of the population, wherein the graphical display includes a comparison of the reduced number of features of the profile with the plurality of features associated with the plurality of clusters of portions of the population.

5. The method of claim 1 wherein the profile is associated with a patient, the plurality of features relate to healthcare data associated with the patient, and the probability score is a phenotype prediction.

6. The method of claim 1 wherein visually presenting the comparison includes presenting at least one of at least one of a radar chart, a sliding scale graph, or a distribution graph.

7. The method of claim 1 wherein the profile is grouped in the cluster based on the profile contribution values and the contribution values of the portion of the population being similar to each other within a threshold.

8. The method of claim 1 wherein determining the probability score includes making a statistical comparison between at least one feature associated with the profile and a corresponding feature for the portion of the population.

9. A system for explaining an output from a machine learning model, the system comprising:
an interface for at least receiving from a machine learning model:
a probability score associated with a profile,
a plurality of features associated with the profile, and
a contribution value for each of the plurality of features that represents each feature's contribution to the probability score; and
at least one processor executing instructions stored on memory and configured to:
transform the plurality of features to a reduced number of features of based at least on the contribution value of each of the plurality of features,
generate at least one cluster of a portion of a population, wherein the cluster is generated based on:
a probability score associated with the portion of the population,
a plurality of features associated with the portion of the population,
a contribution value for each of the plurality of features associated with the portion of the population;
group the profile in the generated cluster based on the profile contribution values and the contribution values of the portion of the population; and
visually present to a user a comparison of the reduced number of features of the profile with the plurality of features associated with the portion of the population to explain the probability score.

10. The system of claim 9 further comprising a user interface for graphically presenting the probability score to the user.

11. The system of claim 10 wherein the visual presentation includes a graphical comparison of the reduced number of features of the profile with the plurality of features associated with the portion of the population.

12. The system of claim 11 wherein the processor is further configured to generate a plurality of clusters of portions of the population, wherein the graphical display includes a comparison of the reduced number of features of the profile with the plurality of features associated with the plurality of clusters of portions of the population.

13. The system of claim 9 wherein the profile is associated with a patient, the plurality of features relate to healthcare data associated with the patient, and the probability score is a phenotype prediction.

14. The system of claim 9 wherein the comparison is visually presented as at least one of a radar chart, sliding scale graph, or a distribution graph.

15. The system of claim 9 wherein the at least one processor groups the profile in the cluster based on the profile contribution values and the contribution values of the portion of the population being similar to each other within a threshold.

16. The system of claim 9 wherein the processor determines the probability score by making a statistical comparison between at least one feature associated with the profile and a corresponding feature for the portion of the population.

17. A computer program product for explaining an output from a machine learning model, the computer program product comprising computer executable code embodied in one or more non-transitory computer readable media that, when executing on one or more processors, performs the steps of:
receiving from a machine learning model:
a probability score associated with a profile,
a plurality of features associated with the profile, and
a contribution value for each of the plurality of features that represents each feature's contribution to the probability score;
transforming the plurality of features to a reduced number of features of based at least on the contribution value of each of the plurality of features;
generating at least one cluster of a portion of a population, wherein the cluster is generated based on:
a probability score associated with the portion of the population,
a plurality of features associated with the portion of the population, and
a contribution value for each of the plurality of features associated with the portion of the population;
grouping the profile in the generated cluster based on the profile contribution values and the contribution values of the portion of the population; and
visually presenting to a user a comparison of the reduced number of features of the profile with the plurality of features associated with the portion of the population to explain the probability score.

18. The computer program product of claim 17 further comprising computer executable code that, when executing on one or more processors, performs the step of graphically presenting the probability score to the user.

19. The computer program product of claim 18 wherein the visual presentation includes a graphical comparison of the reduced number of features of the profile with the plurality of features associated with the portion of the population.

20. The computer program product of claim 19 further comprising computer executable code that, when executing on one or more processors, performs the step of generating a plurality of clusters of portions of the population, wherein the graphical display includes a comparison of the reduced number of features of the profile with the plurality of features associated with the plurality of clusters of portions of the population.
